SEQUENCE LISTING (19 sequences)

SEQ ID NO: 1 - protein, 320 aa, Escherichia coli
MIKKIGVLTSGGDAPGMNAAIRGVVRSALTEGLEVMGIYDGYLGLYEDRMVQLDRYSVSDMINRGGTFLGSARFPEFRDENIRAVAIENLKKRGIDALVVIGGDGSYMGAMRLTEMGFPCIGLPGTIDNDIKGTDYTIGFFTALSTVVEAIDRLRDTSSSHQRISVVEVMGRYCGDLTLAAAIAGGCEFVVVPEVEFSREDLVNEIKAGIAKGKKHAIVAITEHMCDVDELAHFIEKETGRETRATVLGHIQRGGSPVPYDRILASRMGAYAIDLLLAGYGGRCVGIQNEQLVHHDIIDAIENMKRPFKGDWLDCAKKLY

SEQ ID NO: 2 - DNA, 963 bp, Escherichia coli
atgattaagaaaatcggtgtgttgacaagcggcggtgatgcgccaggcatgaacgccgcaattcgcggggttgttcgttctgcgctgacagaaggtctggaagtaatgggtatttatgacggctatctgggtctgtatgaagaccgtatggtacagctagaccgttacagcgtgtctgacatgatcaaccgtggcggtacgttcctcggttctgcgcgtttcccggaattccgcgacgagaacatccgcgccgtggctatcgaaaacctgaaaaaacgtggtatcgacgcgctggtggttatcggcggtgacggttcctacatgggtgcaatgcgtctgaccgaaatgggcttcccgtgcatcggtctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggagatctgacgttggctgcggccattgccggtggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgatctgctgctggcaggttacggcggtcgttgtgtaggtatccagaacgaacagctggttcaccacgacatcatcgacgctatcgaaaacatgaagcgtccgttcaaaggtgactggctggactgcgcgaaaaaactgtattaa

SEQ ID NO: 3 - DNA, 39 bp, Artificial Sequence
  Synthetic PFKA primer
cgtacccggggatccatgattaagaaaatcggtgtgttg

SEQ ID NO: 4 - DNA, 39 bp, Artificial Sequence
  Synthetic PFKA primer
gactctagaggatccttaatacagttttttcgcgcagtc

SEQ ID NO: 5 - DNA, 33 bp, Artificial Sequence
  Synthetic F1 forward primer
cggcgtagaggatcaggagcttatcgactgcac

SEQ ID NO: 6 - DNA, 28 bp, Artificial Sequence
  Synthetic F1 reverse primer
ccggcgtagagaatccacaggacgggtg

SEQ ID NO: 7 - DNA, 27 bp, Artificial Sequence
  Synthetic F2 forward primer
ctgtggattctctacgccggacgcatc

SEQ ID NO: 8 - DNA, 29 bp, Artificial Sequence
  Synthetic F2 reverse primer
aagggcatcggtcgtcgctctcccttatg

SEQ ID NO: 9 - DNA, 3576 bp, Artificial Sequence
  Synthetic pTSa-EX1 vector
gaattcagccagcaagacagcgatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatcacctgtaagtcggacgttccgatcacctgtaacgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcaagaacatgtgagcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatttttaaggcagttattggtgcccttaaacgcctggttgctacgcctgaataagtgataataagcggatgaatggcagaaattc

SEQ ID NO: 10 - DNA, 40 bp, Artificial Sequence
  Synthetic primer
cttgatatcgaattcttctcatgtttgacagcttatcatc

SEQ ID NO: 11 - DNA, 36 bp, Artificial Sequence
  Synthetic primer
gggctgcaggaattcgaatttctgccattcatccgc

SEQ ID NO: 12 - DNA, 37 bp, Artificial Sequence
  Synthetic primer
cttgatatcgaattaggcctgtcatcgtctatatacg

SEQ ID NO: 13 - DNA, 42 bp, Artificial Sequence
  Synthetic primer
cgtgttgttcgaattcgatggatattcctccagtatcatgtg

SEQ ID NO: 14 - DNA, 32 bp, Artificial Sequence
  Synthetic primer
catcgaattcgaacaacacgccgatgtatgac

SEQ ID NO: 15 - DNA, 40 bp, Artificial Sequence
  Synthetic primer
acatgagaagaattgacagatccggtcagttcacattatc

SEQ ID NO: 16 - DNA, 39 bp, Artificial Sequence
  Synthetic primer
cagaaattcgaattgcgatcatcaccaaccaggaaattc

SEQ ID NO: 17 - DNA, 37 bp, Artificial Sequence
  Synthetic primer
gggctgcaggaattgggtatttcaggcggcagtaaag

SEQ ID NO: 18 - DNA, 40 bp, Artificial Sequence; identical to SEQ ID NO: 10
  Synthetic primer

SEQ ID NO: 19 - DNA, 36 bp, Artificial Sequence; identical to SEQ ID NO: 11
  Synthetic primer

(19) United States
(12) Patent Application Publication
(10) Pub. No.: US 2018/0142274 A1
(43) Pub. Date: May 24, 2018

(54) MICROORGANISM OF GENUS KOMAGATAEIBACTER HAVING ENHANCED CELLULOSE PRODUCTIVITY, METHOD OF PRODUCING CELLULOSE USING THE SAME, AND METHOD OF PRODUCING THE MICROORGANISM

(71) Applicant: Samsung Electronics Co., Ltd., Suwon-si (KR)

(72) Inventors: Soonchun Chung, Seoul (KR); Jiae Yun, Hwaseong-si (KR); Jinkyu Kang, Hwaseong -si (KR); Jinhwan Park, Suwon-si (KR)

(21) Appl. No.: 15/820,231

(22) Filed: Nov. 21, 2017

(30) Foreign Application Priority Data
Nov. 21, 2016 (KR) 10-2016-0154878

(51) Int. Cl. C12P 19/04; C12N 9/24

(52) U.S. Cl. CPC C12P 19/04; C12Y 207/01011; C12N 9/2402

(57) ABSTRACT

Provided are a microorganism of genus Komagataeibacter having enhanced cellulose productivity and yield, a method of producing cellulose by using the microorganism, and a method of producing the microorganism.

### CROSS-REFERENCE TO RELATED APPLICATION

This application claims the benefit of Korean Patent Application No. 10-2016-0154878, filed on Nov. 21, 2016, in the Korean Intellectual Property Office, the disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 12,502 Byte ASCII (Text) file named “729030_ST25.TXT,” created on Nov. 21, 2017.

### BACKGROUND

### 1. Field

The present disclosure relates to a microorganism of genus Komagataeibacter having enhanced cellulose productivity, a method of producing cellulose by using the same, and a method of producing the microorganism.

### 2. Description of the Related Art

Cellulose produced by cultured microorganisms exists as a primary structure of β-1,4 glucans composed of glucose, which form a network structure of fibril bundles. This cellulose is also called ‘biocellulose’ or ‘microbial cellulose’.

Unlike plant cellulose, microbial cellulose is pure cellulose entirely free of lignin or hemicellulose. Microbial cellulose is 100 nm or less in width, and has increased water absorption and retention capacity, increased tensile strength, increased elasticity, and increased heat resistance, when compared to plant cellulose. Due to these improved characteristics, microbial cellulose is useful in a variety of fields, such as cosmetics, medical products, dietary fibers, audio speaker diaphragms, and functional films.

Therefore, to meet the demands for microbial cellulose, there is a need to produce microorganisms having enhanced cellulose productivity. This invention provides such a microorganism.

### SUMMARY

An aspect of the disclosure provides a microorganism of genus Komagataeibacter comprising a genetic modification that increases the activity of phosphofructose kinase (PFK) and enhances cellulose productivity.

Another aspect of the disclosure provides a method of producing cellulose by using a Komagataeibacter microorganism comprising a genetic modification that increases the activity of phosphofructose kinse (PFK).

Still another aspect of the disclosure provides a method of producing a Komagataeibacter microorganism comprising a genetic modification that increases the activity of phoshpofructose kinase (PFK).

### BRIEF DESCRIPTION OF THE DRAWINGS

These and/or other aspects will become apparent and more readily appreciated from the following description of the embodiments, taken in conjunction with the accompanying drawings in which:

FIG. 1 shows cellulose nanofiber (CNF) production of K. xylinus strain into which a pfkA gene is introduced;

FIG. 2 shows CNF yield of a K. xylinus strain into which a pfkA gene is introduced;

FIG. 3 shows CNF production and yield of a K. xylinus strain into which a pfkA gene is introduced during fermentation in a medium free of carboxy methyl cellulose (CMC); and

FIG. 4 shows CNF production and yield of a K. xylinus strain into which a pfkA gene is introduced during fermentation in a medium including CMC.

### DETAILED DESCRIPTION

The term “increase in activity” or “increased activity”, or similar terms, as used herein, may refer to a detectable increase in an activity of a cell, a protein, or an enzyme. The “increase in activity” or “increased activity” or the like may also refer to an activity level of a modified (e.g., genetically engineered) cell, protein, or enzyme that is higher than that of a comparative cell, protein, or enzyme of the same type, such as a cell, protein, or enzyme that does not have a given genetic modification (e.g., original or “wild-type” cell, protein, or enzyme). For example, an activity of a modified or engineered cell, protein, or enzyme may be increased by about 5% or more, about 10% or more, about 15% or more, about 20% or more, about 30% or more, about 50% or more, about 60% or more, about 70% or more, or about 100% or more than an activity of a non-engineered cell, protein, or enzyme of the same type, i.e., a wild-type or “parent” cell, protein, or enzyme. A cell having an increased activity of a protein or an enzyme may be identified by using any method known in the art.

An increase in an activity of an enzyme or a polypeptide may be achieved by an increase in expression or specific activity. The increase in expression may be caused by introduction of an exogenous polynucleotide encoding the enzyme or the polypeptide into a cell (e.g., increase of the copy number thereof) or by modification of a regulatory region of the polypeptide.

The polynucleotide encoding the enzyme may be operably linked to a regulatory sequence that allows expression thereof, for example, a promoter, a polyadenylation site, or a combination thereof. The polynucleotide whose copy number is increased may be endogenous or heterologous. The endogenous gene refers to a gene a copy of which is included in a microorganism prior to introducing the genetic modification (e.g., native gene). The term “heterologous” means that the gene is “foreign” or “not native” to the species. In either case, a polynucleotide or gene that is externally introduced into a cell is referred to as “exogenous,” and an exogenous gene or polynucleotide may be homologous or heterologous with respect to a host cell into which the gene is introduced. Thus, the microorganism into which the polynucleotide encoding the enzyme is introduced may be a microorganism that already includes the gene encoded by the polynucleotide (e.g., the gene or polynucleotide is endogenous to the microorganism). Alternatively, the microorganism can be without a copy of the gene prior to its introduction (e.g., the polynucleotide or gene is heterologous to the microorganism)

The term “increase in the copy number” of a gene may be caused by introduction of an exogenous gene or by amplification of a gene already existing in the microorganism. An increase in copy number encompasses the introduction of an exogenous gene that does not exist in the non-engineered cell (i.e., prior to introduction of the exogenous gene). The introduction of the gene may be mediated by a vehicle such as a vector. The introduction may be a transient introduction in which the gene is not integrated into a genome, or may be an introduction that results in integration of the gene into the genome. The introduction may be performed, for example, by introducing a vector into the cell, the vector including a polynucleotide encoding a target polypeptide, and then, replicating the vector in the cell, or by integrating the polynucleotide into the genome.

The introduction of the gene may be performed via a known method, for example, transformation, transfection, or electroporation. The gene may be introduced via a vehicle or as it is. The term “vehicle” or “vector”, as used herein, refers to a nucleic acid molecule that is able to deliver other nucleic acids linked thereto into a cell. As a nucleic acid sequence mediating introduction of a specific gene, the vehicle used may be a vector, a nucleic acid construct, or a cassette. The vector may include, for example, a plasmid vector or a viral vector (e.g., plasmid or viral expression vector), such as a replication-defective retrovirus, adenovirus, adeno-associated virus, or a combination thereof.

The term “parent cell” refers to an original cell, for example, a non-genetically engineered cell of the same type as an engineered microorganism. With respect to a particular genetic modification, the “parent cell” may be a cell that lacks the particular genetic modification, but is identical in all other respects. Thus, the parent cell may be a cell that is used as a starting material to produce a genetically engineered microorganism having an increased activity of a given protein (e.g., a protein having a sequence identity of about 90% or higher with respect to phosphofructose kinase (PFK)). In addition, with respect to a microorganism having an enhanced activity of PFK in a cell due to genetic modification of a gene encoding PFK, the parent cell may be a microorganism that is not genetically modified. The same comparison is also applied to other genetic modifications.

The term “gene”, as used herein, refers to a nucleic acid fragment encoding a particular protein, and may or may not include a regulatory sequence of a 5′-non coding sequence and/or a 3′-non coding sequence.

The term “sequence identity” of a polynucleotide or a polypeptide, as used herein, refers to a degree of identity between bases or amino acid residues of sequences obtained after the sequences are aligned so as to best match in certain comparable regions. The sequence identity is a value that is measured by comparing two sequences in certain comparable regions via optimal alignment of the two sequences, in which portions of the sequences in the certain comparable regions may be added or deleted compared to reference sequences. A percentage of sequence identity may be calculated by, for example, comparing two optimally aligned sequences in the entire comparable regions, determining the number of locations in which the same amino acids or nucleic acids appear to obtain the number of matching locations, dividing the number of matching locations by the total number of locations in the comparable regions (that is, the size of a range), and multiplying a result of the division by 100 to obtain the percentage of the sequence identity. The percentage of the sequence identity may be determined using a known sequence comparison program, for example, BLASTN (NCBI), BLASTP (NCBI), CLC Main Workbench (CLC bio), MegAlign™ (DNASTAR Inc), etc.

Various levels of sequence identity may be used to identify various types of polypeptides or polynucleotides having the same or similar functions or activities. For example, the sequence identity may include a sequence identity of about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100%.

The term “genetic modification”, as used herein, refers to an artificial alteration in a constitution or structure of a genetic material of a cell.

An aspect of the disclosure provides a microorganism of genus Komagataeibacter including a genetic modification that increases phosphofructose kinase (PFK) enzyme activity. In some embodiments, the microorganism has enhanced (increased) cellulose productivity as compared to the same Komagataeibacter microorganism without the genetic modification that increases the activity of PFK.

PFK is a protein that phosphorylates fructose-6-phosphate into fructose-1,6-bisphosphate, and exists as a homotetramer in bacteria and mammals and as an octomer in yeast. PFK may be PFK1 (also, referred to as “PFKA”). PFK1 may belong to the enzyme classified as EC 2.7.1.11. PFK may be from bacteria. PFK may be from genus Escherichia, genus Bacillus, genus Mycobacterium, genus Zymomonas, or genus Vibrio. PFK may be derived from E. coli, for example, E. coli MG1655.

PFK may catalyze conversion of ATP and fructose-6-phosphate into fructose-1,6-bisphosphate and ADP. PFK may be allosterically activated by ADP and diphosphonucleoside and allosterically inhibited by phosphoenolpyruvate. PFK may be a polypeptide having a sequence identity of about 90% or higher, about 95% or higher, or about 100% with an amino acid sequence of SEQ ID NO: 1

In the microorganism, the genetic modification may increase expression of a gene encoding PFK. The genetic modification may be an increase of the copy number of a gene encoding PFK or a modification of an expression regulatory sequence of a gene encoding the PFK. The increase of the copy number may be caused by introduction of an exogenous gene into the cell or by amplification of an endogenous gene. The gene may be a polynucleotide encoding PFK1 that belongs to the enzyme classified as EC 2.7.1.11. The PFK may be from bacteria. The gene may be from genus Escherichia, genus Bacillus, genus Mycobacterium, genus Zymomonas, or genus Vibrio. The gene may be from E. coli. The gene may have a nucleotide sequence encoding an amino acid sequence having a sequence identity of about 90% or more, 95% or more, or 99% or more with the amino acid sequence of SEQ ID NO: 1. The gene may have a sequence identity of about 90% or more, about 95% or more, or about 99% or more of a nucleotide sequence of SEQ ID NO: 2.

The genetic modification may introduce the gene encoding the PFK, for example, via a vehicle such as a vector. The gene encoding the PFK once introduced may exist within or outside the chromosome (i.e., may be integrated into the bacterial chromosome or expressed from an extra-chromosomal construct). Furthermore, a plurality of PFK genes (which may be the same or different) can be introduced, for example, 2 or more, 5 or more, 10 or more, 30 or more, 50 or more, 100 or more, or 1000 or more genes encoding PFK.

The microorganism may be any species of Komagataeibacter that produces bacterial cellulose, for instance K. xylinus (also, referred to as “G. xylinus”), K. rhaeticus, K. swingsii, K. kombuchae, K. nataicola, or K. sucrofermentans. The strain may be one that lacks endogenous PFK activity.

Another aspect of the disclosure provides a method of producing cellulose, the method including culturing the microorganism of genus Komagataeibacter comprising a genetic modification that increases a PFK activity, as disclosed herein, in a medium to produce cellulose; and collecting the cellulose from a culture. All aspects of the microorganism of genus Komagataeibacter used in the method are as described above with respect to the microorganism itself.

The culturing may be performed in a medium containing a carbon source, for example, glucose. The medium used for culturing the microorganism may be any general medium suitable for host cell growth, such as a minimal or complex medium containing appropriate supplements. The suitable medium may be commercially available or prepared by a known preparation method.

The medium may be a medium that may satisfy the requirements of a particular microorganism depending on a selected product of culturing. The medium may be a medium including components selected from the group consisting of a carbon source, a nitrogen source, a salt, trace elements, and combinations thereof.

The culturing conditions may be appropriately controlled for the production of cellulose. The culturing may be performed under aerobic conditions for cell proliferation. The culturing may be performed by spinner culture or static culture without shaking. A density of the microorganism may be a density which gives enough space so as not to disturb production of cellulose.

The term “culture conditions”, as used herein, mean conditions for culturing the microorganism. Such culture conditions may include, for example, a carbon source, a nitrogen source, or an oxygen condition utilized by the microorganism. The carbon source that may be utilized by the microorganism may include monosaccharides, disaccharides, or polysaccharides. The carbon source may include glucose, fructose, mannose, or galactose as an assimilable glucose. The nitrogen source may be an organic nitrogen compound or an inorganic nitrogen compound. The nitrogen source may be exemplified by amino acids, amides, amines, nitrates, or ammonium salts. An oxygen condition for culturing the microorganism may be an aerobic condition of a normal oxygen partial pressure or a low-oxygen condition including about 0.1% to about 10% of oxygen in the atmosphere. A metabolic pathway may be modified in accordance with a carbon source or a nitrogen source that may be actually used by a microorganism.

The medium may include ethanol or cellulose. The ethanol may be about 0.1 to 5% (v/v), for example, about 0.3 to 2.5% (v/v), about 0.3 to 2.0% (v/v), about 0.3 to 1.5% (v/v), about 0.3 to 1.25% (v/v), about 0.3 to 1.0% (v/v), about 0.3 to 0.7% (v/v), or about 0.5 to 3.0% (v/v) with respect to a volume of the medium. The cellulose may be about 0.5 to 5% (w/v), about 0.5 to 2.5% (w/v), about 0.5 to 1.5% (w/v), or about 0.7 to 1.25% (w/v) with respect to a volume of the medium. The cellulose may be carboxylated cellulose. The cellulose may be CMC. The CMC may be sodium CMC.

The method may include separating the cellulose from the culture. The separating may be, for example, collecting of a cellulose pellicle formed on the top of the medium. The cellulose pellicle may be collected by physically stripping off the cellulose pellicle or by removing the medium. The separating may be collecting of the cellulose pellicle while maintaining its shape without damage.

Another aspect of the disclosure provides a method of producing a microorganism having enhanced cellulose productivity, the method including introducing a gene encoding a PKF into a microorganism of genus Komagataeibacter. The introducing of a gene encoding a PKF may comprise introducing a vehicle (e.g., vector) including the gene into the microorganism. In the method, the genetic modification may include amplifying the gene, engineering a regulatory sequence of the gene, or engineering a sequence of the gene itself. The engineering may be insertion, substitution, conversion, or addition of a nucleotide. All other aspects of the method are as previously described with respect to the microorganism itself.

Hereinafter, the present invention will be described in more detail with reference to Examples. However, these Examples are provided for illustrative purposes only, and the invention is not intended to be limited by these Examples.

### Example 1. Preparation of K. xylinus Including Phosphofructose Kinase (PFK) Gene and Production of Cellulose

In this Example, an exogenous PFK gene was introduced into Komagataeibacter xylinus DSM2325 (DSM, Germany), and the microorganism was cultured to examine the effects of the gene introduction on cellulose productivity.

1. Preparation of Vector for Over-Expressing pfkA

The phosphofructose kinase (pfk) gene in K. xylinus was introduced by homologous recombination. The specific procedure is as follows:

An amplification product was obtained by PCR amplification using a pTSa-EX1 vector (SEQ ID NO: 9) as a template and a set of primers of SEQ ID NO: 5 and SEQ ID NO: 6 and a set of primers of SEQ ID NO: 7 and SEQ ID NO: 8. The amplification product was cloned by using an In-Fusion GD cloning kit (Takara) at the BamHI and Sail restriction sites of the pTSa-EX1 vector. The pTSa-EX1 vector is a shuttle vector which replicates in both E. coli and X. xylinus.

In order to introduce pfkA by homologous recombination, an open reading frame (ORF) (SEQ ID NO: 2) of the pfkA gene was produced by PCR amplification using a genome DNA of E. coli K12 MG1655 as a template and a set of primers of SEQ ID NO: 3 and SEQ ID NO: 4 as primers. Fragments of the pfkA gene were cloned at the BamHI and Sail restriction enzyme sites of the pTSa-EX11 vector by using an In-Fusion GD cloning kit (Takara) to prepare vector pTSa-Ec.pfkA for over-expressing pfkA.

2. Preparation of Vector for Inserting E. coli pfkA Gene

A tetA gene was amplified by PCR amplification using a pTSa-Ec.pfkA vector as a template and SEQ ID NO: 10 and SEQ ID NO: 11 as a set of primers. The PCR product was cloned at a EcoRI restriction enzyme site of a pMSK+ vector (Genbank Accession No. KJ922019) by using an In-fusion GD cloning kit (Takara) to prepare a pTSK+ vector.

A homologous region of a site to which a pfkA gene was about to be inserted was amplified by PCR using a genome DNA of K. xylinus as a template and each of primer sets of SEQ ID NOS: 12 and 13, SEQ ID NOS: 14 and 15, and SEQ ID NOS: 16 and 17 as primers, and the amplification product was cloned at an EcoRI restriction enzyme site of a pTSK+ vector by using an In-fusion GD cloning kit (Takara) to prepare a pTSK-(del)2760 vector.

A Ptac::Ec.pfkA gene was amplified by PCR amplification using the pTSa-Ec.pfkA vector as a template and a primer set of SEQ ID NO: 18 and SEQ ID NO: 19 as primers. The PCR product was cloned at an EcoRI restriction enzyme site of a pTSK-(del)2760 vector by using an In-fusion GD cloning kit (Takara) to prepare a pTSK-(del)2760-Ec.pfkA vector.

3. Introduction of Phosphofructose Kinase Gene

In order to introduce a nucleotide sequence of SEQ ID NO: 2, which is a pkfA gene of E. coli, to K. xylinus, a cassette for inserting a Ptac::Ec.pfkA gene was amplified using the pTSK-(del)2760-Ec.pfkA vector as a template and a primer set of SEQ ID NO: 12 and SEQ ID NO: 17 as primers, and the amplification product was introduced to a K. xylinus strain by the following transformation procedure.

More specifically, the K. xylinus strain was spread on an HS-agar medium (0.5% peptone, 0.5% yeast extract, 0.27% Na₂HPO₄, 0.15% citric acid, 2% glucose, and 1.5% bacto-agar) supplemented with 2% of glucose, and then cultured at 30° C. 3 days. The strain was inoculated in a 5 ml HS medium supplemented with 0.2% (v/v) of cellulase (sigma, Cellulase from Trichoderma reesei ATCC 26921), and then cultured at 30° C. 2 days. A cell suspension thus cultured was inoculated in a 100 ml HS medium supplemented with 0.2% (v/v) of cellulose so that a cell density (OD600) was 0.04, and then the resultant was cultured at 30° C. so that a cell density was 0.4 to 0.7. The cultured strain was washed with 1 mM of HEPES buffer, washed three times with 15% of glycerol, and re-suspended with 1 ml of 15% of glycerol to prepare a competent cell.

100 μl of the competent cell thus prepared was transferred to 2 mm of an electro-cuvette, 3 μg of the Ptac::Ec.pfkA cassette was added thereto, and a vector was introduced to the competent cell by electroporation (2.4 kV, 200Ω, 25 ρF). The vector-introduced cell was re-suspended in 1 ml of a HS medium containing 2% of glucose and 0.1% (v/v) cellulose, transferred to a 14 ml round-bottom tube, and cultured at 30° C. and 160 rpm for 16 hours. The cultured cell was spread on a HS medium supplemented with 2% glucose, 1% ethanol, and 5 μg/ml of tetracycline and cultured at 30° C. for 4 days. Strains having a tetracycline resistance were selected to prepare PFK gene-over-expressing strains.

Strains transformed with the vector but without the Ptac::Ec. pfkA cassette (referred to as Δ2760) served as an additional control.

4. Glucose Consumption and Cellulose and Gluconate Productions

The designated K. xylinus strains were inoculated into 50 ml of a HS medium supplemented with 5% glucose and 1% of ethanol, and the resultant was stirred and cultured at 30° C. at 230 rpm for 5 days. Then, glucose consumption and the product cellulose were quantified. Glucose and gluconate were analyzed by using HPLC equipped with the Aminex HPX-87H column (Bio-Rad, USA). The product of cellulose was quantified by measuring a weight after washing the cellulose solid produced in the flask with 0.1 N sodium hydroxide solution and distilled water, and freeze-drying the resultant. A gluconate yield was analyzed.

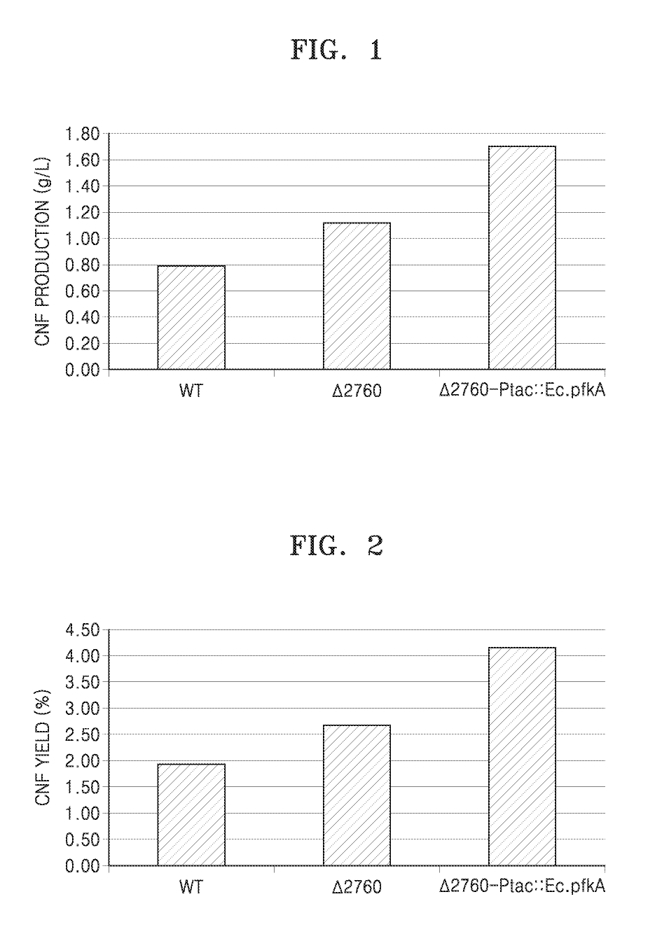

The results are shown in FIGS. 1 to 3. FIG. 1 shows a CNF product obtained from a culture cultured from a K. xylinus strain introduced with a PFK gene. As shown in FIG. 1, when the PFKA gene was introduced to K. xylinus, the CNF production increased about 115% with respect to a wild-type strain. Table 1 illustrates the data shown in FIGS. 1 and 2.

TABLE 1

| | Glucose | Gluconate | | Gluconate | CNF |
|---|---|---|---|---|---|
| | consumption | production | CNF | yield | yield |
| | (g/L) | (g/L) | (g/L) | (%) | (%) |
| WT | 40.17 | 29.11 | 0.79 | 72.46 | 1.96 |
| Δ2760 | 41.65 | 31.71 | 1.12 | 76.13 | 2.68 |
| Δ2760- | 40.76 | 29.85 | 1.70 | 73.24 | 4.18 |
| Ptac::Ec.pfkA | | | | | |

FIG. 2 shows a yield of cellulose nanofibers (CNFs) obtained from the culture prepared by culturing the each of the K. xylinus strains introduced with the PFK gene. As shown in FIG. 2, when the PFKA gene was introduced to K. xylinus, the CNF yield increased about 113% with respect to a wild-type strain.

CNF production in the wild-type and recombinant strains was also analyzed by fermentation culture. Briefly, the strains were spread on a HSD medium (5 g/L yeast extract, 5 g/L bactopeptone, 2.7 g/L Na₂HPO₄, 1.15 g/L citric acid, and 20 g/L glucose) and a plate containing 20 g/L agar, and the resultant was cultured at 30° C. for 3 days.

Starter fermentation was performed by adding 100 mL of a HSD medium in a 250 mL flask, inoculating 3 loops of microorganism, and culturing the resultant at 30° C. at 150 rpm for 20 hours.

Main fermentation was performed by using a 1.5 L bench-type fermentor (GX2-series, Biotron) system, a baffle was removed, and a stirring environment with enhanced vertical movement was formed by using a pitch-type impeller and a microsparger.

Operation conditions included an initial volume of 0.7 L, a temperature of 30° C., pH 5.0 (adjusted by using a neutralizing agent 3 N KOH (aq)), a stirring rate of 150 rpm, an airflow amount of 0.7 L/min, a medium, which was a HS medium supplemented with 40 g/L glucose, and inoculation at a rate of 14% (v/v).

In the CMC-added environment, fermentation evaluation included adding Na_CMC 1.0% (w/v) to the same HS medium and changing the stirring rate to 250 rpm from the conditions described above. CNF quantity was measured based on weight after pre-treating the collected fermentation solution, that is washing the collected fermentation solution with a 0.1 N NaOH (aq) solution at 90° C. for 2 hours.

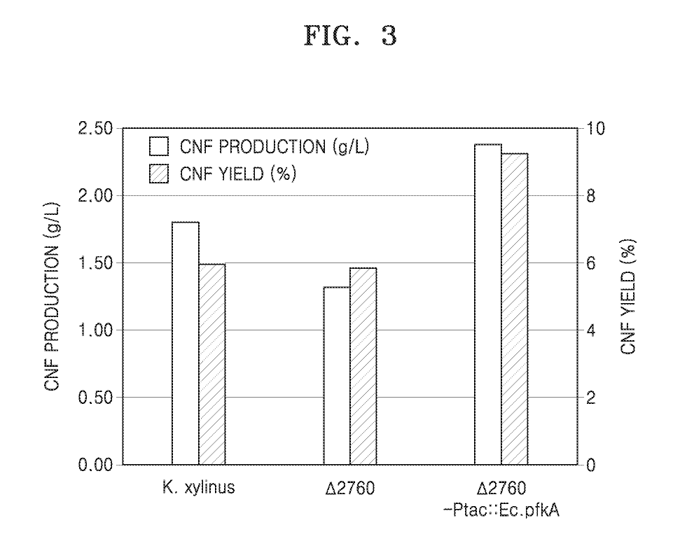

FIG. 3 shows CNF production and yield when a K. xylinus strain into which a pfkA gene is introduced was cultured by fermentation. As shown in FIG. 3, when the pfkA gene was introduced to K. xylinus, the CNF productions increased about 32%, and the CNF yields increased about 55% than those of the control group. The yield is a percent ratio of the CNF weight produced with respect to a weight of glucose used in the fermentation.

TABLE 2

| | Glucose | | |
|---|---|---|---|
| | consumption | CNF production | CNF yield |
| CMC free fermentation | (g/L) | (g/L) | (%) |
| WT | 29.70 | 1.80 | 5.95 |
| Δ2760 | 22.50 | 1.32 | 5.85 |
| Δ2760-Ptac::Ec.pfkA | 25.20 | 2.38 | 9.25 |

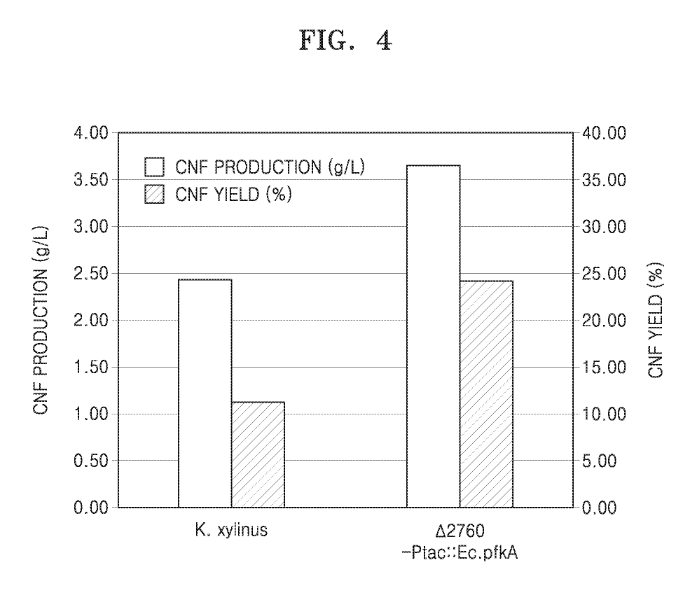

FIG. 4 shows CNF production and yield when a K. xylinus strain into which a pfkA gene is introduced was supplemented with CMC and fermented. As shown in FIG. 4, when the pfkA gene was introduced to K. xylinus, the CNF productions increased about 50%, and the CNF yields increased about 116% than those of the control group. Table 3 illustrates the data shown in FIG. 4.

TABLE 3

| | Glucose | CNF | CNF |
|---|---|---|---|
| CMC added | consumption | production | yield |
| fermentation | (g/L) | (g/L) | (%) |
| WT | 21.7 | 2.43 | 11.18 |
| Δ2760-Ptac::Ec.pfkA | 15.1 | 3.65 | 24.15 |

This indicates that the introduced exogenous pfkA phosphorylated fructose-6-phosphate of the strain into fructose-1,6-bisphosphate and thus influenced the corresponding reaction and cellulose production.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A Komagataeibacter microorganism comprising a genetic modification that increases phosphofructose kinase (PFK) enzyme activity and enhances cellulose productivity.

2. The microorganism of claim 1, wherein the genetic modification is an increase of the copy number of a gene encoding PFK or a modification of an expression regulatory sequence of a gene encoding PFK.

3. The microorganism of claim 2, wherein the increase of the copy number is caused by introduction of an exogenous gene encoding PFK.

4. The microorganism of claim 1, wherein the PFK is an enzyme classified as EC 2.7.1.11.

5. The microorganism of claim 1, wherein PFK is an enzyme having a sequence identity of about 90% or higher with an amino acid sequence of SEQ ID NO: 1.

6. The microorganism of claim 2, wherein the PFK is an enzyme classified as EC 2.7.1.11.

7. The microorganism of claim 2, wherein the PFK is a enzyme having a sequence identity of 90% or higher with an amino acid sequence of SEQ ID NO: 1.

8. The microorganism of claim 1, wherein the microorganism comprises a gene encoding PFK from Escherichia, Bacillus, Mycobacterium, genus Zymomonas, or genus Vibrio.

9. The microorganism of claim 2, wherein the microorganism comprises a gene encoding PFK from Escherichia, Bacillus, Mycobacterium, Zymomonas, or Vibrio.

10. The microorganism of claim 2, wherein the gene encoding PFK has the nucleotide sequence of SEQ ID NO: 2.

11. The microorganism of claim 1, wherein the Komagataeibacter is Komagataeibacter xylinus.

12. A method of producing cellulose, the method comprising:
culturing the Komagataeibacter microorganism of claim 1; and
collecting cellulose from the culture.

13. The method of claim 12, wherein the genetic modification increases the copy number of a gene encoding the PFK or a modification of an expression regulatory sequence of a gene encoding the PFK.

14. The method of claim 13, wherein the increase of the copy number is caused by introduction of an exogenous gene encoding PFK.

15. The method of claim 13, wherein the PFK is from Escherichia, Bacillus, Mycobacterium, Zymomonas, or Vibrio.

16. The method of claim 13, wherein PFK is a polypeptide having a sequence identity of 90% or higher with an amino acid sequence of SEQ ID NO: 1.

17. The method of claim 13, wherein the genetic modification increases the copy number of a gene encoding a polypeptide that has a sequence identity of about 90% or higher with an amino acid sequence of SEQ ID NO: 1 or modifies an expression regulatory sequence of a gene encoding a polypeptide that has a sequence identity of about 90% or higher with an amino acid sequence of SEQ ID NO: 1.

18. The method of claim 12, wherein the Komagataeibacter is Komagataeibacter xylinus.

19. The method of claim 12, wherein the medium comprises about 0.5% (w/v) to about 5.0% (w/v) of carboxymethylcellulose (CMC), about 0.1% (v/v) to about 5.0% (v/v) of ethanol, or about 0.5% (w/v) to about 5.0% (w/v) of CMC and about 0.1% (v/v) to about 5.0% (v/v) of ethanol.

20. A method of producing a Komagataeibacter microorganism having enhanced cellulose productivity, the method comprising introducing a gene encoding a PKF into a Komagataeibacter microorganism.
